(19) United States
(12) Patent Application Publication
(10) Pub. No.: US 2016/0193211 A1
(43) Pub. Date: Jul. 7, 2016

(54) COMBINATIONS OF A BTK INHIBITOR AND FLUOROURACIL FOR TREATING CANCERS

(71) Applicant: BIONSIL S.R.L. IN LIQUIDAZIONE, Milano (IT)

(72) Inventors: Marialuisa LAVITRANO, Milano (IT); Emanuela GRASSILLI, Monza (IT); Roberto GIOVANNONI, Milano (IT); Fabio PISANO, Monsterace (IT); Gabriele ROMANO, Germignaga (IT); Laura MASIERO, Bresso (IT); Maria Grazia CERRITO, Milano (IT)

(21) Appl. No.: 14/909,675

(22) PCT Filed: Aug. 4, 2014
(86) PCT No.: PCT/EP2014/066724

§ 371 Date: Feb. 2, 2016

(30) Foreign Application Priority Data
Aug. 2, 2013 (EP) 13179049.5

(51) Int. Cl. A61K 31/519; A61K 31/513

(52) U.S. Cl. CPC A61K 31/519; A61K 31/513

(57) ABSTRACT

The present application describes therapies for the effective treatment of colon and colorectal carcinomas. The present invention relates to a pharmaceutical kit, comprising combinations of a BTK inhibitor and fluorouracil, for the treatment of colon and colorectal carcinomas also in the case in which such carcinomas are drug resistant and therefore allows to overcome cancer drug resistance.

### FIELD OF THE INVENTION

The present invention describes novel therapies for the effective treatment of drug resistant cancers, for example colon and colorectal carcinomas, and therapies for minimizing drug resistance in cancer patients.

### STATE OF THE ART

Apoptosis is a common mode of eukaryotic cell death that is triggered by an inducible cascade of biochemical events leading to the activation of DNA cleavage. Most chemotherapeutic agents exert their anticancer activity by inducing apoptosis. Cell resistance to apoptosis is therefore a major factor which limits the effective anticancer therapy.

Many cancers develop resistance to chemotherapy drugs, a major factor in the failure of many forms of chemotherapy. Drug resistance affects patients with a variety of blood cancers and solid tumors, including breast, ovarian, lung, and lower gastrointestinal tract cancers.

Bruton's tyrosine kinase (BTK), a member of the Tec family of cytoplasmic tyrosine kinases, is intimately involved in multiple signal-transduction pathways regulating survival, activation, proliferation, and differentiation of B-lineage lymphoid cells. There are a number of BTK inhibitor in the clinic. The only molecule that is currently approved is ibrutinib (1-(3-(4-amino-3-(4-phenoxyphenyl)-1H-pyrazolo[3,4-d]pyrimidin-1-yl)piperidin-1-yl)prop-2-en-1-one disclosed in WO2008/039218). Ibrutinib is currently used to treat mantel cell lymphoma (MCL) in patients who have received at least one prior therapy. That is to ibrutinib may be considered a cancer adjuvant therapy, that is additional treatment given after the primary treatment to lower the risk that the cancer will come back. The primary treatment may for example be chemotherapy, radiotherapy, surgery or a combination thereof.

The treatment regime for ibrutinib is a 140 mg capsule taken orally four times a day until unacceptable toxicity or disease progression.

WO2008/110624 describes the identification and isolation of a novel isoform of the BTK protein, having a molecular weight of 65-68 kDa. The novel BTK protein, also identified as p65BTK, is shorter than the BTK identified in B cells, and its mRNA has a different first exon.

It is now emerging that cancers treated with BTK inhibitors, for example irreversible BTK inhibitors have the potential to become resistant to the BTK inhibitor.

### SUMMARY OF THE INVENTION

The present inventors have found that cancer patients can be effectively treated by a therapeutically effective amount of combination therapy comprising a small molecule BTK inhibitor (such as an irreversible BTK inhibitor) and fluorouracil concomitantly, wherein the BTK inhibitor is not adjuvant therapy.

Surprisingly the present investors have established that the combination of a small molecule BTK inhibitor, such as ibrutinib specifically with fluorouracil is beneficial because it provides syngerstic results, which may not be available when the BTK inhibitor is used in combination with other chemotherapy, agents, for example in one embodiment the dose BTK inhibitor, the dose of fluorouracil or the dose of both agents can be reduced in comparison to monotherapy, whilst effective anti-cancer activity, such killing of cancer cells is maintained.

The present invention further concerns a pharmaceutical kit for use in the treatment of cancer. In particular, the pharmaceutical kit is for simultaneous, sequential and separate use of an effective dose of ibrutinib and an effective dose of fluorouracil, in the treatment of both drug-sensitive and drug-resistant cancer.

The reduction of dose is important to patients because it reduces undesirable side effects and therefore increases the tolerance for the therapy. Adverse events associated with BTK inhibitors include thrombocytopenia, diarrhoea, neutropenia, anemia, fatigue, muscloskeletal, pain, peripheral edema, upper respiratory tract infection, nausea, bruising, dyspnea, constipation, rash, abdominal pain, vomiting and decreased appetite.

Thus in one embodiment the BTK inhibitor dose employed is less than the dose for monotherapy (currently 540 mg per day for ibrutinib), for example 10, 15, 20, 25, 30, 35, 40, 45, 50, 55, 60, 65, 70, 75, 80, 85, 90 or 95% of the dose employed in monotherapy.

In one embodiment the dose of ibrutinib is in the range 1 to 60 mg/Kg, for example 5 to 50 mg/kg, such as 10 to 50 mg/Kg, in particular 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 45, 46, 47, 48 and 49 mg/Kg per administration.

In one embodiment the dose of ibrutinib is in the range of from 25 mg/day to 840 mg/day, for example 50, 75, 100, 125, 150, 175, 200, 225, 250, 270, 275, 300, 325, 350, 375, 400, 425, 450, 500, 525, 540, 550, 575 and 600 mg/day.

In one embodiment the dose employed of BTK inhibitor has reduced side effects associated therewith.

What is more providing a lower dose of the BTK inhibitor may result in a lower propensity for the cancer cell to become resistant to the BTK inhibitor. Thus, the development of drug resistance may be avoidable by using a lower dose of the BTK inhibitor, for example 10 to 90% of the monotherapy dose, such as 15 to 50% thereof. Thus in one embodiment there is provide a method of reducing or minimising the development of drug resistance of a cancer cell to a BTK inhibitor, by using the combination of the present disclosure, with a reduced dose of a BTK inhibitor.

Whilst not wishing to be bound by theory the data, particularly in p53 deficient cell lines may suggest that as the BTK inhibitor dose employed increases so does the propensity of the cell to be resistant thereto, see for example FIG. 3B.

Fluorouracil, also known as 5FU reduces the white blood cell counts and renders patients more susceptible to opportunistic infection. In one embodiment the dose employed is less than the dose employed for monotherapy. Various monotherapy protocols exist, for example 500 mg/square meter IV on Days 1 to 5, or 450-600 mg/square meter IV weekly, or 200-400 mg/square meter IV continuous infusion, wherein the dose does not exceed 800 mg/day. In the in vivo studies performed the present inventions doses as low 60 mg per kilo (given as oral gavage) showed synergistic effects with the BTK inhibitor.

In one embodiment the dose of fluorouracil is in the range 10 to 60 mg/Kg such as 15 to 50 mg/Kg, in particular 16, 17, 18, 19, 20, 21, 22, 23, 24, 25, 26, 27, 28, 29, 30, 31, 32, 33, 34, 35, 36, 37, 38, 39, 40, 41, 42, 43, 45, 46, 47, 48 and 49 mg/Kg per administration.

In one embodiment the dose of fluorouracil is in the range of from 400 mg/m2 to 600 mg/m2 per day, for example wherein the dose does not exceed 800 mg per day.

The ability to reduce the 5FU dose is particularly beneficial because it may reduce the amount of white blood cells depleted and thereby increase the patient's ability to fight infection.

Treatment with 5FU shows a significant amount of inter-patient variability. The data generated by the present inventors advantageously suggests that inter-patient variability is reduced with the combination therapy of the present disclosure.

In one embodiment the cancer is a drug resistant cancer, for example a chemotherapy resistant cancer. In one embodiment the drug resistance is a BTK inhibitor.

In one embodiment the cancer is a solid tumor, for example an epithial derived cancer, colorectal cancer, hepatoma (liver cancer), prostate cancer, pancreatic cancer, breast cancer, ovarian cancer, thyroid cancer, renal cancer, bladder cancer, head and neck cancer or lung cancer, such as pancreatic cancer or colorectal cancer.

In one embodiment the cancer is metastatic cancer.

In one embodiment the cancer is a p53 deficient cancer.

In one embodiment the cancer is a leukemia, for example ALL, CLL, CML, mantel cell lymphoma or AML.

In one embodiment the BTK inhibitor is an irreversible BTK inhibitor. In one embodiment the BTK inhibitor is selected from the group comprising ibrutinib, HM-71224, BGB-3111, CG-036806, CC-292, ACP-196, GDC-0834, ONO-4049, RN-486, SNS-062, TAS-5567, AVL-101, AVL-291, PCI-45261, HCI-1684, LFM-A13 and PLS-123, for example ibrutinib.

In one embodiment the BTK inhibitor is not LFM-A13.

In one aspect there is provided a method of treating a fluorouracil resistant cancer patient comprising administering a therapeutically effective amount of a BTK inhibitor (such as an irreversible BTK inhibitor), for sensitizing the patient to treatment with fluorouracil.

Also provided is a combination therapy comprising a BTK inhibitor (such as an irreversible BTK inhibitor) and fluorouracil for concomitant administration, wherein the BTK inhibitor is not adjuvant therapy, for use in the treatment of cancer, for example leukemia or a solid tumor, such as cancer of epithelial origin as described herein. In one embodiment the cancer is drug resistant. In one embodiment the cancer is p53 deficient.

In a further aspect there is provided use of

Use of comprising a BTK inhibitor is an irreversible BTK inhibitor. In one embodiment the BTK is a compound disclosed herein, for example ibrutinib, HM-71224, BGB-3111, CG-036806, CC-292, ACP-196, GDC-0834, ONO-4049, RN-486, SNS-062, TAS-5567, AVL-101, AVL-291, PCI-45261, HCI-1684 and PLS-123, in particular ibrutinib) and fluorouracil in the manufacture of a combination therapy for concomitant administration, for the treatment of cancer, for example leukemia or a solid tumor, such as cancer of epithelial origin as described herein. In one embodiment the cancer is drug resistant. In one embodiment the cancer is p53 null.

In further aspect there is provided use of a BTK inhibitor, for example an irreversible BTK inhibitor for example ibrutinib, in the manufacture of a medicament for sensitizing cancer to chemotherapy, such a fluorouracil therapy. In one aspect the present inventors have surprisingly found that if you administer ibrutinib and fluorouracil to colon carcinomas and to colorectal carcinomas, said carcinomas are treated.

Therefore, in one aspect the present invention concerns a pharmaceutical kit for example, for use in the treatment of colon and colorectal carcinomas.

In particular the invention relates to a pharmaceutical kit for simultaneous, sequential and separate use of an effective dose of ibrutinib and an effective dose of fluorouracil, in the treatment of colon and colorectal carcinomas, both drug-sensitive and drug-resistant.

The invention further relates to a method for the treatment of resistant colon and colorectal carcinomas, comprising the administration of and effective dose of ibrutinib and of an effective dose of fluorouracil.

As will be further described in the detailed description of the invention, the pharmaceutical kit of the present invention has the advantages of being specific for the treatment of colon and colorectal carcinomas, both drug-sensitive and drug-resistant.

### BRIEF DESCRIPTION OF THE DRAWINGS

The characteristics and advantages of the present invention will be apparent from the detailed description reported below, from the Examples given for illustrative and non-limiting purposes, and from the annexed FIGS. 1-10, wherein:

FIG. 1 shows the effect of different concentrations of Ibrutinib on the proliferation of three colon carcinoma cell lines which are resistant to 5FU (HCT116p53KO, SW480 and DLD-1) and on the sensitive HCT116 cell line as illustrated in particular in the growth curves (short term assays) as described in Example 1:

FIG. 1a: dose-response curve of HCT116 cells grown in presence of increasing concentrations of ibrutinib (from 0 to 10 μM), for 72 hours; 5000 cells/well were seeded in triplicates and cell number was assessed each 24 hs by use of CellTiter 96 AQueous Non-Radioactive Cell Proliferation Assay (Promega).

FIG. 1b: dose-response curve of HCT116p53KO cells grown in presence of increasing concentrations of ibrutinib (from 0 to 10 μM) for 72 hours; 5000 cells/well were seeded in triplicates and cell number was assessed each 24 hs by use of CellTiter 96 AQueous Non-Radioactive Cell Proliferation Assay (Promega).

FIG. 1c: dose-response curve of DLD-1 cells grown in presence of increasing concentrations of ibrutinib (from 0 to 10 μM) for 72 hours; 5000 cells/well were seeded in triplicates and cell number was assessed each 24 hs by use of CellTiter 96 AQueous Non-Radioactive Cell Proliferation Assay (Promega).

FIG. 1d: dose-response curve of SW480 cells grown in presence of increasing concentrations of ibrutinib (from 0 to 10 μM) for 72 hours; 5000 cells/well were seeded in triplicates and cell number was assessed each 24 hs by use of CellTiter 96 AQueous Non-Radioactive Cell Proliferation Assay (Promega).

FIG. 2 shows the effect of different concentrations of Ibrutinib (from 0 to 20 μM) on cloning efficiency and on the long term proliferation of three colon carcinoma cell lines which are resistant to 5FU (HCT116p53KO, SW480 and DLD-1) and on the sensitive HCT116 cell line as illustrated in the colony assay experiment (long term assay) described in Example 2. Cells were seeded at low density (1000 cell/each cell line) and grown for 10-12 days in the presence of concentrations of ibrutinib ranging from 0 to 20 μM. Medium was replaced each 3 days and the end of the treatment colonies were visualized by crystal violet staining.

FIG. 3 shows the effect of different concentrations of Ibrutinib (from 0 to 20 μM) and of ibrutinib (from 0 to 20 μM)+5FU (200 μM) on viability of HCT116 and HCT116p53KO colon carcinoma cell lines as described in Example 3. HCT116p53KO and HCT116 cell lines were grown in vitro for 72 hs in the presence of different concentrations of ibrutinib (from 0 to 20 μM)±5FU (200 μM) and their viability was evaluated at the end of the incubation using the calcein assay.

FIG. 4 shows the effect of the combination of 20 μM Ibrutinib with 200 μM 5FU on drug-sensitive colon cancer cells (HCT116), drug-sensitive colon cancer cells overexpressing p65BTK (HCT116p65BTK) and drug-resistant colon cancer cells (HCT116p53KO) as described in Example 3. The three cell lines were grown in vitro for 72 hs in the presence of 20 μM ibrutinib+200 μM 5FU and their viability was evaluated at the end of the incubation using the calcein assay.

FIG. 5 shows the effect of the combination of Ibrutinib with low doses of 5FU on drug-sensitive colon cancer cells (HCT116).

FIG. 5a shows representative photographs of HCT116p53KO cells taken at 72 hours of incubation in presence of 200 μM 5FU, 20 μM Ibrutinib, 200 μM 5FU+20 μM Ibrutinib as described in Example 4.

FIG. 5b: shows representative photographs of HCT116 cells taken at 72 hours of incubation in presence of 200 μM 5FU, 10 μM 5FU, 20 μM Ibrutinib, 10 μM 5FU+20 μM Ibrutinib as shows representative photographs of HCT116 cells taken at 72 hours of incubation in presence of 10 μM 5FU, 20 μM ibrutinib, 10 μM 5FU+20 μM ibrutinib. For better comparison, treatment with the maximally effective (on drug-sensitive cells) concentration of 200 μM 5FU is shown side to side with the combination 10 μM 5FU+20 μM Ibrutinib as described in Example 4.

FIG. 5c shows the dose-response curve on HCT116. Cell lines were grown in vitro for 72 hs in the presence of different low concentrations of ibrutinib (from 0 to 10 μM) and of ibrutinib (from 0 to 10 μM)+a low dose of 5FU (10 μM); viability was evaluated at the end of the incubation using the crystal violet staining method. 100% represent the percentage of living cells at day 0, before starting the treatment with the effect of different low concentrations of Ibrutinib (from 0 to 10 μM)+a low dose of 5FU (10 μM) on viability of HCT116 colon carcinoma cell lines as described in Example 4.

FIG. 6 shows drug-resistant SW480 colon cancer cells treated for 72 hs in absence of any drug or in presence of Ibrutinib 20 μM; 5FU 200 μM; Ibrutinib 20 μM+5FU 200 μM; oxaliplatin 20 μM; Ibrutinib 20 μM+oxaliplatin 20 μM, as described in Example 5. Viability was evaluated at the end of the incubation using the calcein assay.

FIG. 7 shows the results of the short and long-term combined treatment of ibrutinib and three different anti-tumoural “targeted” drugs for the inhibition of EGFR (cetuximab, panitumumab) and of VEGF (bevacizumab) on the HCT116p53KO and HCT116 cell lines as described in Example 5. Upper: HCT116p53KO and HCT116 cell lines were grown in vitro for 72 hs in the presence of 10 μg/ml cetuximab, 75 μg/ml panitumumab, 25 μg/ml bevacizumab±Ibrutinib 20 μM and their viability was evaluated by Trypan blue staining 72 hs after the combined treatment. Lower: Cells were seeded at low density (1000 cell/each cell line) and grown for 10-12 days in the presence of 10 μg/ml cetuximab, 75 μg/ml panitumumab, 25 μg/ml bevacizumab±Ibrutinib 20 μM. Medium was replaced each 3 days and at the end of the treatment colonies were visualized by crystal violet staining.

FIG. 8 shows the effects of the combined treatment of ibrutinib and cetuximab, or panitumumab or bevacizumab (“targeted” drugs for the inhibition of EGFR) on cell viability of two different drug-resistant colon cancer cell lines (HT29 and SW480) as depicted in Example 5. Cells were left untreated or treated with 25 μg/ml bevacizumab or 10 μg/ml cetuximab or 75 μg/ml panitumumab alone or in combination with 20 mM ibrutinib. Percentage of viable cells was evaluated after 72 hs of treatment by MTT assay.

FIG. 9: shows the kinetics of growth and volumes of HCT116p53KO xenografts upon treatment with 5FU, Ibrutinib low-dose (25 mpk) and combination thereof.

FIG. 9a shows the growth curve of tumors derived from HCT116p53KO cells xenografted in CD1 mice after treatment with Ibrutinib (25 mpk), 5FU (60 mpk) and Ibrutinib (25 mpk)+5FU (60 mpk), compared to the control (vehicle) (n=8 mice/group) as described in Example 6.

FIG. 9b shows the range of tumor volumes measured at the end of the treatment of the mice xenografted with HCT116p53KO cells with Ibrutinib (25 mpk), 5FU (60 mpk), Ibrutinib (25 mpk)+5FU (60 mpk), and vehicle alone (ctrl) (n=8 mice/group). In the box plot graph of FIG. 9b, the bold line indicates the median of the values, the box indicates the first and third quartile. The maximum and minimum values for each group are also reported as described in Example 6.

FIG. 10 shows the kinetics of growth and volumes of HCT116p53KO xenografts upon treatment with 5FU, Ibrutinib high-dose (50 mpk) and combination thereof.

FIG. 10a shows the growth curve of tumors derived from HCT116p53KO cells xenografted in CD1 mice after treatment with Ibrutinib (50 mpk), 5FU (60 mpk) and Ibrutinib (50 mpk)+5FU (60 mpk), compared to the control (vehicle) (n=8 mice/group) as described in Example 6.

FIG. 10b shows the range of tumor volumes measured at the end of the treatment of the mice xenografted with HCT116p53KO cells with Ibrutinib (50 mpk), 5FU (60 mpk), Ibrutinib (50 mpk)+5FU (60 mpk), and vehicle alone (ctrl) (n=8 mice/group). In the box plot graph of FIG. 10b, the bold line indicates the median of the values, the box indicates the first and third quartile. The maximum and minimum values for each group are also reported as described in Example 6.

FIG. 11 Proliferative index and percentage of necrotic area of the tumoral tissues excised from the mice at the end of the different treatments.

FIG. 11a shows the proliferative index, evaluated as the percentage of Ki67-positive cells, of the tumoral tissues excised from the mice at the end of the treatment at the end of the treatment with vehicle alone (Ctrl), 5FU (60 mpk), Ibrutinib 25 mpk, 5FU (60 mpk)+Ibrutinib 25 mpk. Data are expressed as mean±SEM.

FIG. 11b shows the proliferative index, evaluated as the percentage of Ki67-positive cells, of the tumoral tissues excised from the mice at the end of the treatment with vehicle alone (Ctrl), 5FU (60 mpk), Ibrutinib 50 mpk, 5FU (60 mpk)+Ibrutinib 50 mpk. Data are expressed as mean±SEM.

FIG. 11c shows the percentage of necrotic area measured in the tumoral tissues excised from the mice at the end of the treatment with vehicle alone (Ctrl), 5FU (60 mpk), Ibrutinib 25 mpk, Ibrutinib 50 mpk, 5FU (60 mpk)+Ibrutinib 25 mpk, 5FU (60 mpk)+Ibrutinib 50 mpk. Data are expressed as mean±SEM as described in Example 6.

FIG. 12. Kinetics of growth and proliferative index of HCT116 xenografts upon treatment with 5FU, Ibrutinib low-dose (25 mpk) and combination thereof.

FIG. 12a shows the growth curve of tumors derived from HCT116 xenografted in CD1 mice after treatment with Ibrutinib (25 mpk), 5FU (60 mpk) and Ibrutinib (25 mpk)+5FU (60 mpk), compared to the control (vehicle) (n=8 mice/group). The proliferative index of the tumoral tissues excised from the mice at the end of the treatment, evaluated as the percentage of Ki67-positive cells, is shown in FIG. 12b. Data are expressed as mean±SEM as described in Example 6.

FIG. 13. Kinetics of growth and proliferative index of HCT116 xenografts upon treatment with 5FU, Ibrutinib high-dose (50 mpk) and combination thereof.

FIG. 13a shows the growth curve of tumors derived from HCT116 xenografted in CD1 mice after treatment of with Ibrutinib (50 mpk), 5FU (60 mpk) and Ibrutinib (50 mpk)+5FU (60 mpk), compared to the control (vehicle) (n=8 mice/group).

FIG. 13b shows the proliferative index of the tumoral tissues excised from the mice at the end of the treatment evaluated as the percentage of Ki67-positive cells. Data is expressed as mean±SEM as described in Example 6.

FIG. 14. Effect s of the combined treatment of ibrutinib and 5FU on drug-resistant epithelial cancer cells derived from tumours other than colon as illustrated in the experiments described in Example 7.

FIG. 14a. BT549 breast carcinoma cells were left untreated or treated with 200 μM 5FU or 10 μM ibrutinib or the combination of the two. Percentage of viable cells was evaluated after 72 hs of treatment by MTT assay

FIG. 14b. Capan-1 pancreatic carcinoma cells were left untreated or treated with 200 μM 5FU or 10 μM ibrutinib or the combination of the two. Percentage of viable cells was evaluated after 72 hs of treatment by MTT assay

FIG. 14c. BTMKN-45 stomach carcinoma cells were left untreated or treated with 50 μM 5FU or 10 μM ibrutinib or the combination of the two. Percentage of viable cells was evaluated after 72 hs of treatment by MTT assay.

FIG. 15 Effect of different concentrations of ibrutinib on cell viability of 5FU drug-resistant, p53-null (HCT116p53KO, SW480 and HT-29) and sensitive, p53 wild type (HCT116, RKO) colon carcinoma cell lines. Cells were grown for 72 hs in the presence of concentrations of ibrutinib ranging from 0 to 30 μM and viability assessed at the end of the experiment by the calcein assay as illustrated in the experiment described in Example 3

### DETAILED DESCRIPTION OF THE INVENTION

A cancer patient as employed herein is a patient diagnosed or suspected of having cancer.

A combination therapy as employed herein is a therapy where the BTK inhibitor and the chemotherapeutic agent fluorouracil are employed in the same treatment regime.

A small molecule BTK inhibitor as employed herein refers to a chemically synthesized molecule, generally with a molecular weight of 500 Daltons or less, which inhibits the BTK protein, for example irreversibly inhibits the same. A small molecular BTK inhibitor as employed herein is not DNA or RNA molecule.

Example of small molecule BTK inhibitors presently in the clinic include ibrutinib, HM-71224, BGB-3111, CG-036806, CC-292, ACP-196, GDC-0834, ONO-4049, RN-486, SNS-062, TAS-5567, AVL-101, AVL-291, PCI-45261, HCI-1684 and PLS-123

In the present invention, by Ibrutinib or ibru as employed herein is a compound of formula:

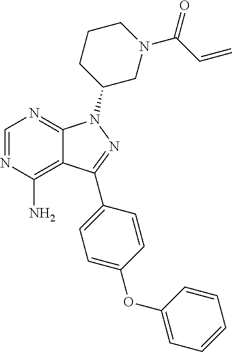 [SMILES: C=CC(=O)N1CCC[C@@H](n2nc(-c3ccc(Oc4ccccc4)cc3)c3c(N)ncnc32)C1]

with the CAS identifier number: 936563-96-1, and chemical IUPAC name: 1-[(3R)-3-[4-amino-3-(4-phenoxyphenyl)pyrazolo[3,4-d]pyrimidin-1-yl]piperidin-1-yl]prop-2-en-1-one.

Ibrutinib has been used in preclinical studies for the treatment of chronic lymphocytic leukemia (CLL), and has been shown to promote apoptosis, inhibit proliferation, and also prevent CLL cells from responding to survival stimuli provided by the microenvironment. Ibrutinib has recently been granted accelerated approval from the FDA to treat mantle cell lymphoma (MCL). Ibrutinib is a selective and irreversible inhibitor of the enzyme Bruton tyrosine kinase (Btk) that forms a covalent bond with a cysteine residue on Btk.

Preclinical and clinical studies suggest that the treatment with ibrutinib has a safe toxicological profile and thus provides a benefit to cancer patients who usually experience the adverse side effects of chemotherapy such as fatigue, loss of appetite, nausea or vomiting.

HM-71224 as employed herein refers to a compound with a structure:

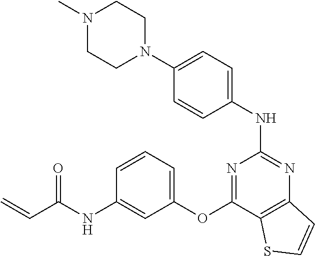 [SMILES: C=CC(=O)Nc1cccc(Oc2nc(Nc3ccc(N4CCN(C)CC4)cc3)nc3ccsc23)c1]

or a derivative thereof, such as

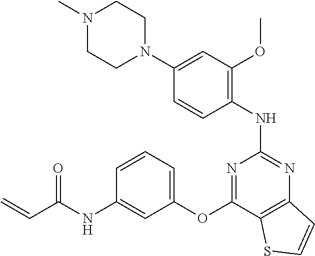 [SMILES: C=CC(=O)Nc1cccc(Oc2nc(Nc3ccc(N4CCN(C)CC4)cc3OC)nc3ccsc23)c1]

for example as disclosed in WO2011/162515 incorporated herein by reference.

Ono-4049 as employed herein refers to the following compound: 6-amino-9-[(3R)-1-(2-butynoyl)-3-pyrrolidinyl]-7-(4-phenoxyphenyl)-7,9-dihydro-8H-purin-8-one.

The compound SNS-062 is disclosed in US application publication no 2012/0157442.

Other BTK inhibitors have the structure shown in Table 1:

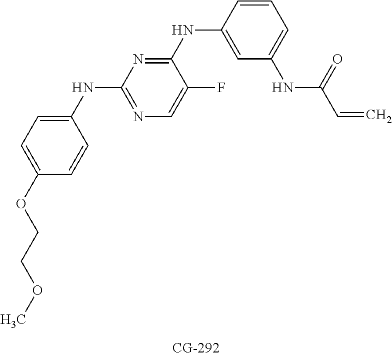 [SMILES: C=CC(=O)Nc1cccc(Nc2nc(Nc3ccc(OCCOC)cc3)ncc2F)c1]

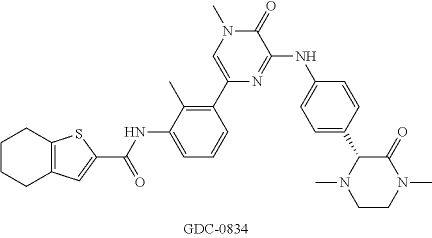 [SMILES: Cc1c(NC(=O)c2cc3c(s2)CCCC3)cccc1-c1cn(C)c(=O)c(Nc2ccc([C@@H]3C(=O)N(C)CCN3C)cc2)n1]

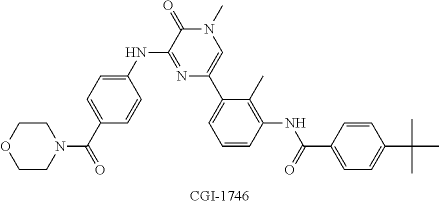 [SMILES: Cc1c(NC(=O)c2ccc(C(C)(C)C)cc2)cccc1-c1cn(C)c(=O)c(Nc2ccc(C(=O)N3CCOCC3)cc2)n1]

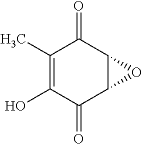

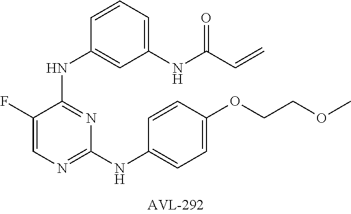 [SMILES: C=CC(=O)Nc1cccc(Nc2nc(Nc3ccc(OCCOC)cc3)ncc2F)c1]

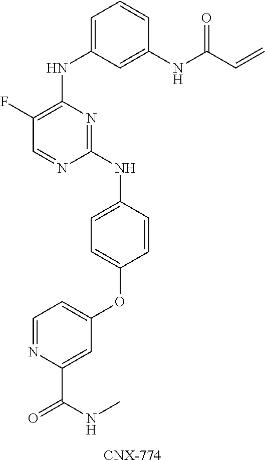

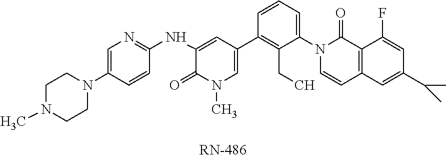 [SMILES: CCc1c(-c2cc(Nc3ccc(N4CCN(C)CC4)cn3)c(=O)n(C)c2)cccc1-n1ccc2cc(C(C)C)cc(F)c2c1=O]

In the present invention, by Fluorouracil or 5-FU is intended an antimetabolite drug used in the treatment of cancer, which acts through the irreversible inhibition of thymidylate synthase. 5-FU induces cell cycle arrest and apoptosis by inhibiting the cell's ability to synthesize DNA. 5-FU has the following IUPAC name: 5-fluoro-1H,3H-pyrimidine-2,4-dione, and CAS number: 51-21-8.

Administered concomitantly as employed herein refers where the pharmacological effects of the both the BTK inhibitor and the fluorouracil co-exist in a patient. Thus the BTK inhibitor may be administered first, at the same time or a short period prior or after administration of the fluorouracil.

In one embodiment the BTK inhibitor is administered as a monotherapy for a period before the fluorouracil dosing in administered. In one embodiment administration of the BTK inhibitor is continued during the phase of fluorouracil treatment or is discontinued shortly before the commencement of the fluorouracil treatment.

Shortly before as employed in this context is a period during which the pharmacological effects of the BTK inhibitor are still present in the patient, for example, 1 hour to 1 week, such as 2 hours to 2 days.

In one embodiment the BTK inhibitor and the fluorouracil are administered within 5 minutes to 1 days of each other, such as within 0.5, 1, 1.5, 2, 2.5, 3, 4, 5, 6, 7, 8, 9, 10, 11, 12, 13, 14, 15, 16, 17, 18, 19, 20, 21, 22 or 23 hours of each other.

In one embodiment administration of the BTK inhibitor is continued after the treatment with combination therapy disclosed herein. In one embodiment the dose of the BTK inhibitor employed in this embodiment is less than 540 mg per day.

Cancer adjuvant therapy where a compound is administered in a separate regime following first line treatment is not concomitant treatment within the meaning of the present application.

In one embodiment the BTK inhibitor is administered orally.

In one embodiment BTK inhibitor is administered intravenously.

In one embodiment the fluorouracil is administered parenterally, for example intravenously.

In one embodiment the fluorouracil is administered orally.

In one embodiment the fluorouracil is administered as a suppository.

In one embodiment the fluorouracil is administered topically, which may be particularly effective for colorectal cancer.

In one embodiment the BTK inhibitor and the fluorouracil are co-formulated, as a solid or liquid formulation, for example as tablet or capsule.

Tumor protein p53, also known as p53, cellular tumor antigen p53, phosphoprotein p53, or tumor suppressor p53, is a protein that in humans is encoded by the TP53 gene. The p53 protein is crucial in multicellular organisms, where it regulates the cell cycle and, thus, functions as a tumor suppressor, preventing cancer. As such, p53 has been described as “the guardian of the genome” because of its role in conserving stability by preventing genome mutation. Hence TP53 is classified as a tumor suppressor gene.

P53 deficient cancer as employed herein refers to a cancer that has reduced p53 activity in comparison to a healthy cell or has no p53 activity.

The present invention also concerns a pharmaceutical kit for simultaneous, sequential and separate use of an effective dose of a BTK inhibitor and an effective dose of fluorouracil, wherein said BTK inhibitor is chosen from the group consisting of a BTK inhibitor described herein, for example ibrutinib, HM-71224, BGB-3111, CG-036806, CC-292, ACP-196, GDC-0834, ONO-4049, RN-486, SNS-062, TAS-5567, AVL-101, AVL-291, PCI-45261, HCI-1684, LFM-A13 and PLS-123. The BTK inhibitor is preferably ibrutinib.

The pharmaceutical kit according to the present invention is effective in the treatment of cancer, wherein said cancer is leukemia or a solid tumor.

The solid tumor can be chosen from the group consisting of colorectal cancer, liver cancer, prostate cancer, pancreatic cancer, breast cancer, ovarian cancer, thyroid cancer, renal cancer, bladder cancer, head and neck cancer, or lung cancer. The solid tumor is preferably a colon or a colorectal carcinoma.

Thus in one aspect the present invention concerns a pharmaceutical kit for simultaneous, sequential and separate use of an effective dose of ibrutinib and an effective dose of fluorouracil, in the treatment of colon and colorectal carcinomas.

The inventors have surprisingly found that the treatment of colon or colorectal carcinomas by the simultaneous or sequential administration of ibrutinib and fluorouracil results in a synergic antitumoral effect compared to the administration of either of the components individually. This surprising synergic antitumoral effect has been seen also in drug-resistant colon and colorectal carcinomas, which are those cancers which develop resistance to chemotherapy drugs. The pharmaceutical kit according to the present invention allows to overcome cancer drug resistance due to the many mechanisms of tumor cell evolution and adaptation.

A further surprising advantage of the present invention relates to the fact that the inventors have found that in the treatment of colon or colorectal carcinomas by the simultaneous or sequential administration of ibrutinib and fluorouracil, a smaller or lower amount of the chemotherapeutic drug can be used and is efficacious, in comparison to the amount of chemotherapeutic drug which is used in the treatment of the same carcinomas. The amount of chemotherapeutic drug, fluorouracil, can be reduced in a range from 5% to 80%, preferably in a range from 10% to 60%, more preferably from 20% to 40%, still more preferably from 25% to 35%.

In a further embodiment the invention relates to the use of an effective dose of a BTK inhibitor and an effective dose of fluorouracil, in the treatment of cancer. In a preferred aspect, the BTK inhibitor is chosen from the group consisting of ibrutinib, HM-71224, BGB-3111, CG-036806, CC-292, ACP-196, GDC-0834, ONO-4049, RN-486, SNS-062, TAS-5567, AVL-101, AVL-291, PCI-45261, HCI-1684, LFM-A13 and PLS-123. In a further preferred aspect said cancer is drug-sensitive or drug resistant and is leukemia or a solid tumor, preferably chosen from the group consisting of colorectal cancer, liver cancer, prostate cancer, pancreatic cancer, breast cancer, ovarian cancer, thyroid cancer, renal cancer, bladder cancer, head and neck cancer, or lung cancer.

The present invention further concerns a pharmaceutical kit for simultaneous, sequential and separate use of an effective dose of ibrutinib and an effective dose of fluorouracil, in the treatment of drug-resistant colon and colorectal carcinomas.

In a second embodiment the pharmaceutical kit of the present invention surprisingly allows the treatment of drug-resistant cancer cells in a way that not only shows a synergic antitumoral effect of ibrutinib and fluorouracil, but also allow the use of lower amounts of fluorouracil, and thus a lower effective dose of the chemotheurapeutic drug, thus avoiding the undesirable and often harmful side-effects that these drugs have.

A further aspect of the present invention is a pharmaceutical kit for simultaneous, sequential and separate use of an effective dose of ibrutinib and an effective dose of fluorouracil, wherein the ibrutinib is in the form of a pharmaceutically acceptable salt.

In a further aspect, the invention provides a pharmaceutical kit comprising an effective dose of ibrutinib and an effective dose of fluorouracil, wherein the effective dose of ibrutinib and the effective dose of 5-fluorouracil are formulated as a pharmaceutical form selected from oral form, parenteral form and rectal form.

Depending on the method/route of administration, dosage forms can be of several types. These include many kinds of liquid, solid, and semisolid dosage forms. Common dosage forms include pill, tablet, or capsule, drink or syrup. The route of administration (ROA) for drug delivery is dependent on the dosage form of the substance.

Various dosage forms may exist for a single particular drug, since different medical conditions can warrant different routes of administration.

Routes of administration can be inhalational, buccal (oral), sublingual, nasal, suppository and parenteral.

In a still further aspect, the invention provides a pharmaceutical kit comprising an effective dose of ibrutinib and an effective dose of fluorouracil, wherein the pharmaceutical form is an oral form, preferably a tablet.

Furthermore, the invention relates to a pharmaceutical kit for simultaneous, sequential and separate use of an effective dose of ibrutinib and an effective dose of fluorouracil, wherein the pharmaceutical form comprises at least one pharmaceutical acceptable excipient. An excipient is a pharmacologically inactive substance formulated with the active ingredient, commonly used to bulk up formulations that contain potent active ingredients (often referred to as “bulking agents,” “fillers,” or “diluents”), to allow convenient and accurate dispensation of a drug substance when producing a dosage form. They also can serve various therapeutic-enhancing purposes, such as facilitating drug absorption or solubility, or other pharmacokinetic considerations.

The selection of appropriate excipients also depends upon the route of administration and the dosage form, as well as the active ingredient and other factors.

In a preferred embodiment, the invention provides a pharmaceutical kit comprising an effective dose of ibrutinib and an effective dose of fluorouracil, wherein the effective dose of ibrutinib is in the range of from 1 mg/day to 2000 mg/day (derived from ClinicalTrials.gov; Study Identifiers: NCT01804686, NCT01589302, NCT01855750, NCT01744691, NCT01105247, NCT00849654) and wherein the effective dose of fluorouracil is in the range of from 100 to 2500 mg/m², preferably form 200 to 1500 mg/m², or from 400 mg/m² to 600 mg/m² (derived from the NCI page for colon cancer treatment for heath professionals: http://www.cancer.gov/cancertopics/pdq/treatment/colon/HealthProfessional/page8).

In a more preferred embodiment the invention provides a pharmaceutical kit wherein the effective dose of ibrutinib is in the range of from 10 mg/day to 1680 mg/day.

In a more preferred embodiment the invention provides a pharmaceutical kit wherein the effective dose of ibrutinib is in the range of from 10 mg/day to 1100 mg/day.

In a more preferred embodiment the invention provides a pharmaceutical kit wherein the effective dose of ibrutinib is in the range of from 20 mg/day to 950 mg/day.
In a still more preferred embodiment, the invention provides a pharmaceutical kit comprising an effective dose of ibrutinib and an effective dose of fluorouracil, wherein the effective dose of ibrutinib is in the range of from 25 mg/day to 840 mg/day.
In a preferred embodiment the invention provides a pharmaceutical kit wherein the effective dose of fluorouracil is in the range of from 450 mg/m² to 550 mg/m².

In a still preferred embodiment the invention provides a pharmaceutical kit wherein the effective dose of fluorouracil is lower, preferably in a range from 150 mg/m² to 400 mg/m². In a further aspect of the present invention the pharmaceutical kit comprises at least one container comprising the effective dose of ibrutinib and at least one container comprising the effective dose of fluorouracil, and an instruction leaflet.

In a still further aspect of the present invention provides the use of an effective dose of ibrutinib and an effective dose of fluorouracil, in the treatment of colon and colorectal carcinomas.

The use according to the present invention advantageously allows for the treatment of colon and colorectal carcinomas, wherein the colon and colorectal carcinomas are drug-resistant.

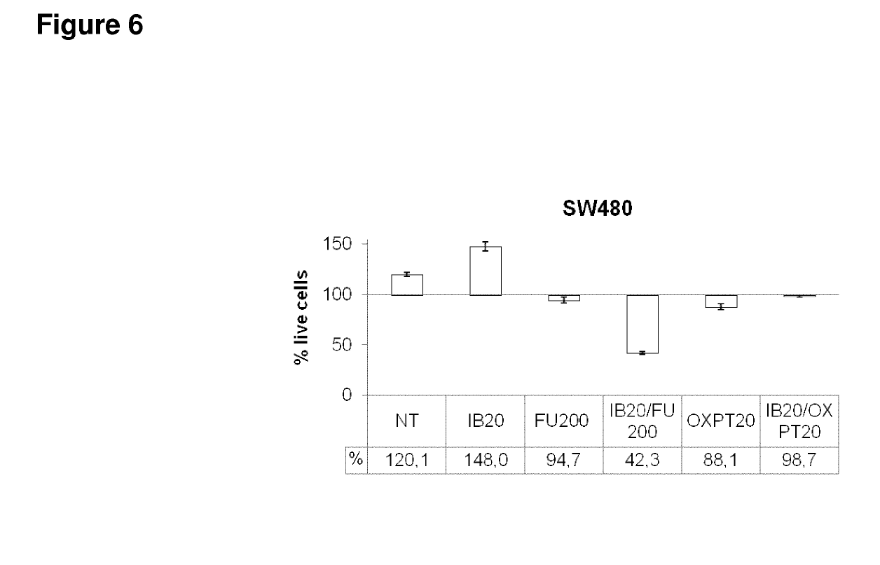

Surprisingly the inventors have found that the effects seen with ibrutinib have not been reproduced oxaliplatin. As will be described in Example 5 and shown in FIG. 6, the evaluation of resistant cancer cell viability at the end of the treatment with, for example oxaliplatin or with the combination of ibrutinib and oxaliplatin, shows that only ibrutinib alone was able to revert the resistance only to 5FU. Reversion of resistance was not obtained with oxaliplatin or with oxaliplatin and ibrutinib.

The invention further relates to a method for the treatment of colon and colorectal carcinomas, or delaying the recurrence of colon and colorectal carcinomas, comprising the administration of and effective dose of ibrutinib and of an effective dose of fluorouracil. The method according to the present invention advantageously allows for the treatment of colon and colorectal carcinomas, wherein the colon and colorectal carcinomas are drug-resistant.

In a first embodiment the method of treatment of a colon or colorectal cancer according to the present invention provides first administering to a patient an effective dose of ibrutinib and sequentially an effective dose of fluorouracil.

In accordance with the present invention the effective doses of ibrutinib and of fluorouracil can be provided in liquid, solid, and semisolid dosage forms depending on the routes of administration as indicated above.

The amount of ibrutinib and of the fluorouracil in the unit dosage form is determined by the dosage to be used on a patient in the methods of the present invention.

In a second embodiment the method comprises administering to a colon or colorectal cancer patient the effective doses of ibrutinib and of fluorouracil simultaneously.
In a further embodiment according to the present invention the effective doses of ibrutinib and of fluorouracil can be administered separately.
In the context of this specification “comprising” is to be interpreted as “including”.
Aspects of the invention comprising certain elements are also intended to extend to alternative embodiments “consisting” or “consisting essentially” of the relevant elements. Any positive embodiment or combination thereof described herein may be the basis of a negative exclusion i.e. a disclaimer.

### Examples

### Example 1

### In Vitro Cell Proliferation Assays

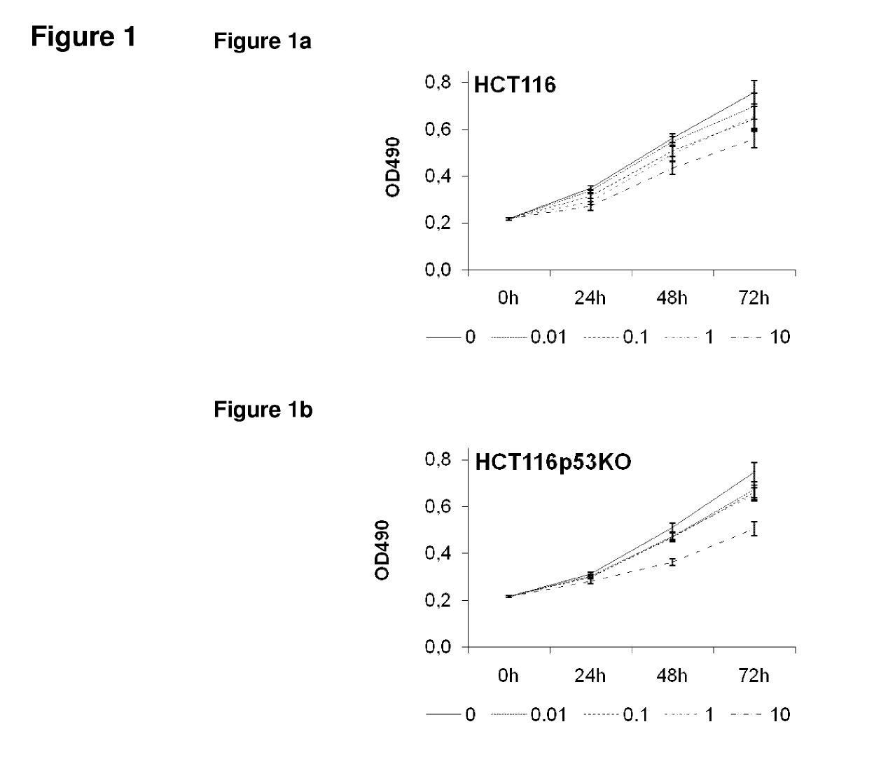

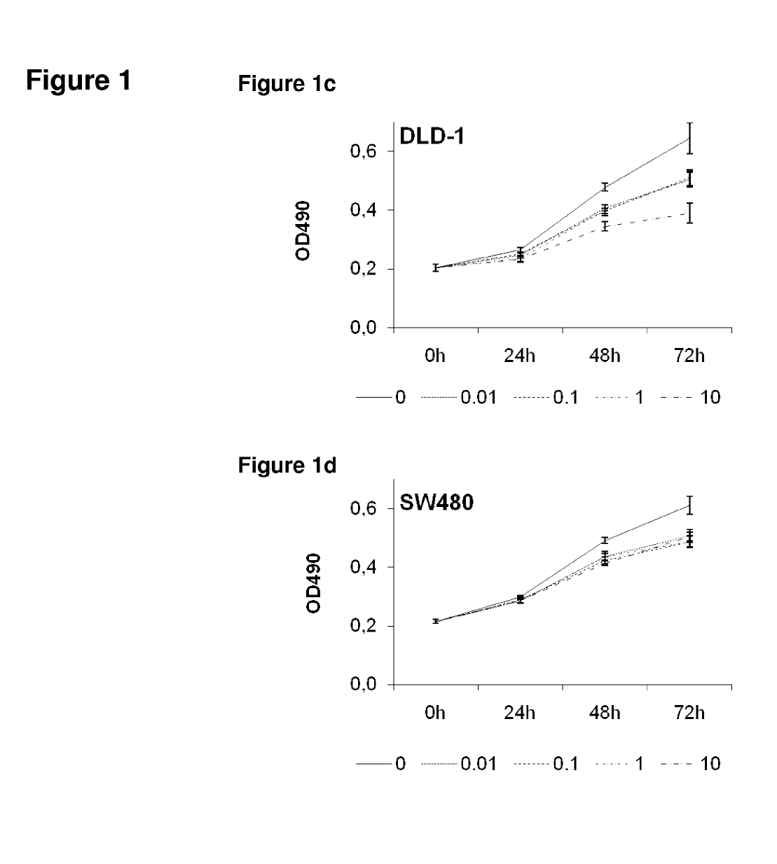

The in vitro proliferation of HCT116p53KO, SW480 and DLD-1 cells, three colon carcinoma cell lines, which are resistant to 5FU treatment, and the HCT116 cell line, which is sensitive to 5FU treatment, were grown and assayed up to 72 h after seeding, in the presence of different concentrations of ibrutinib ranging from 0 to 10 μM.
As can be seen from the graphs of FIG. 1, these short term assays indicate that ibrutinib does not alter the capacity of the cells to proliferate, even at a concentration of 10 μM.

### Example 2

### In Vitro Colony Assays

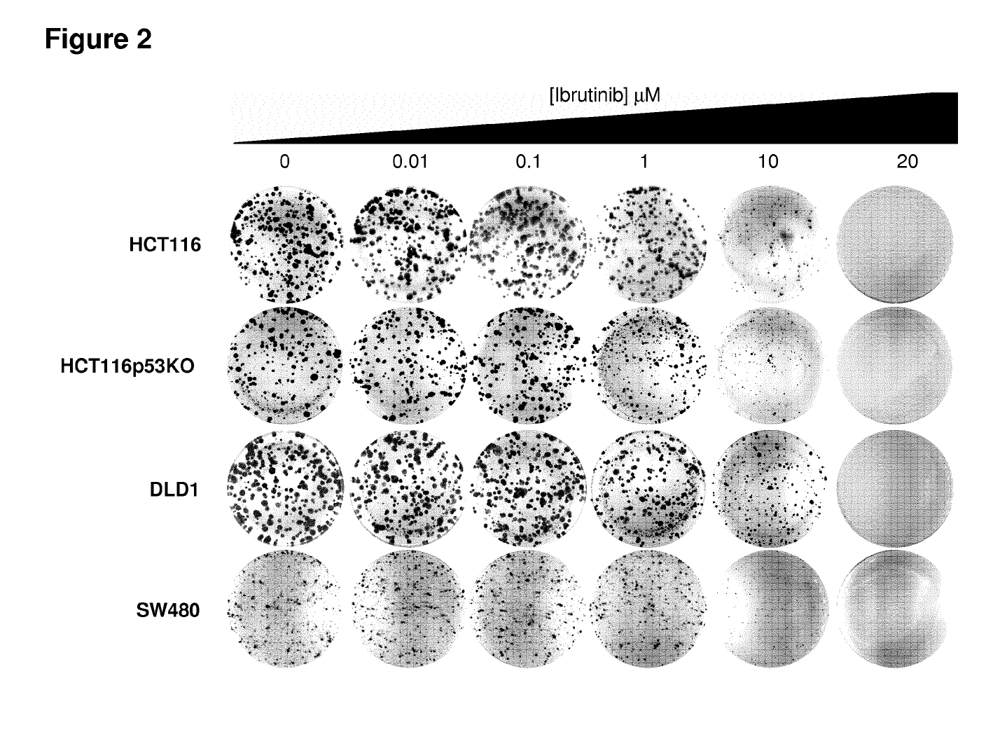

The HCT116p53KO, SW480, DLD-1 cells and HCT116 cell lines were seeded at low density and grown for 10-12 days in the presence of different concentrations of ibrutinib ranging from 0 to 20 μM.
As can be seen from the colony forming assays shown in FIG. 2, these long term assays indicate that even in the long term, treatment with ibrutinib does not affect clonogenic and proliferative capabilities up to a concentration of 1 μM. A concentration of 10 μM Ibrutinib decreases the number and the size of the colonies indicating that it affects both clonogenicity and proliferation, which are eventually inhibited at a concentration of 20 μM.

### Example 3

### Effect of Ibrutinib and Fluorouracil Treatment on Cell Viability

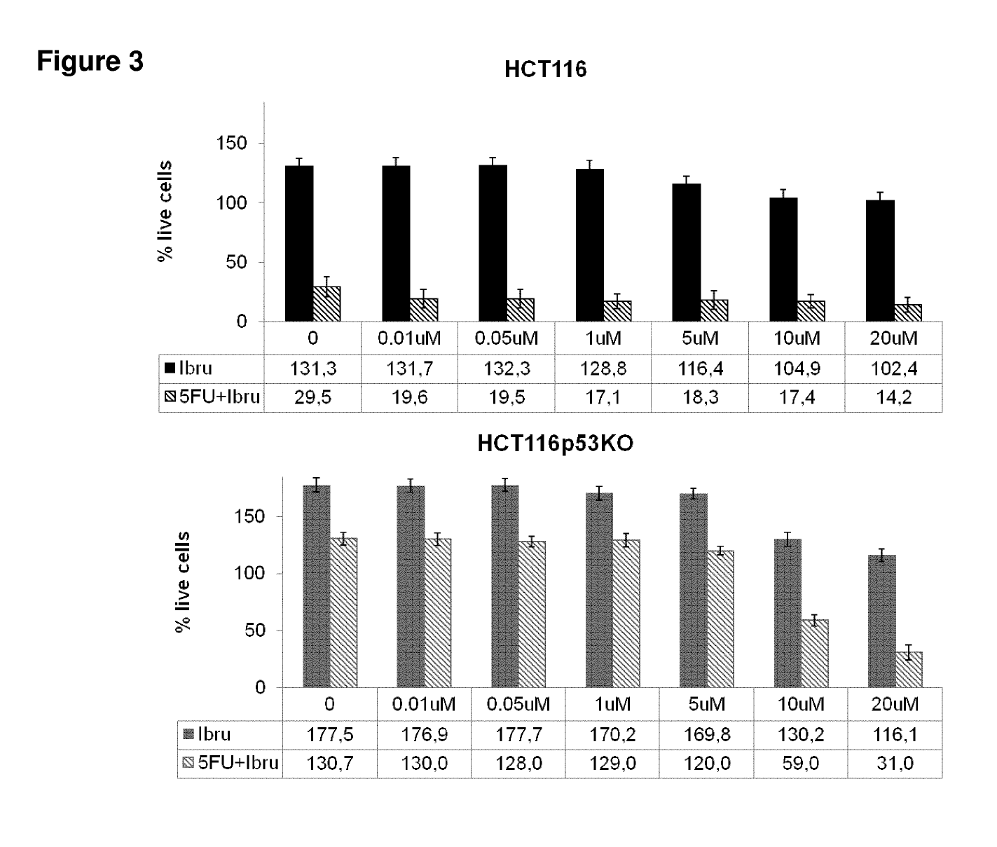

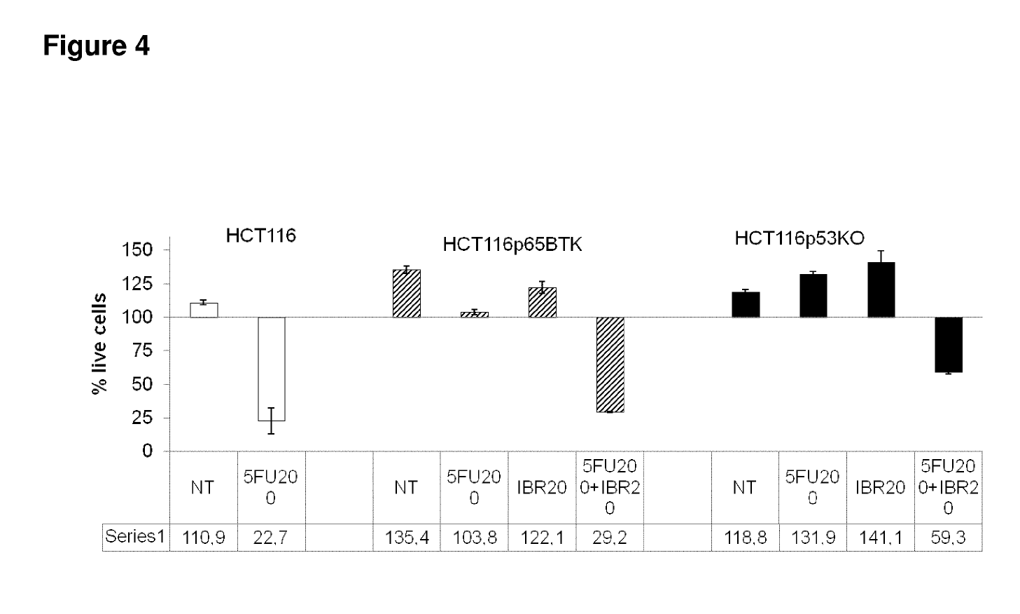

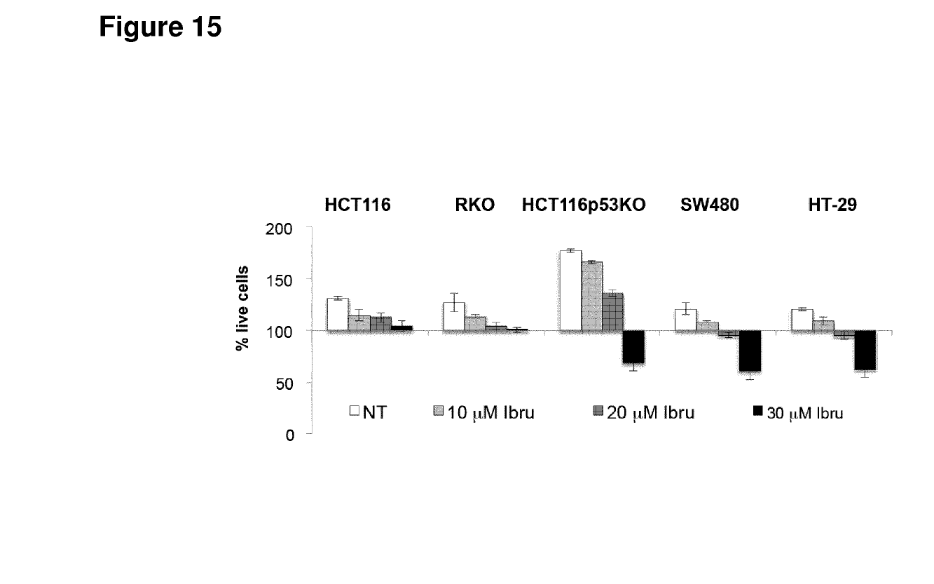

To determine the concentration of Ibrutinib to be used in combination with 5FU drug-resistant, p53-null (HCT116p53KO, SW480 and HT-29) and sensitive, p53 wild type (HCT116, RKO) colon carcinoma cell lines were grown in vitro for 72 hs in the presence of increasing concentrations of ibrutinib (from 0 to 30 μM) and their viability was evaluated at the end of the incubation using the calcein assay (a non fluorescent dye that become fluorescent upon cleavage by lysosomal esterases, active only in living cells). 100% represent the percentage of living cells at day 0, before starting the treatment. As is evident from the graphs shown in FIG. 15, up to 20 μM ibrutinib decreases the viability in a dose-dependent manner in all cell lines; at the highest concentration ibrutinib is preferentially toxic for 5FU drug-resistant, p53-null (HCT116p53KO, SW480 and HT-29).
HCT116p53KO and HCT116 cell lines were grown in vitro for 72 hs in the presence of different concentrations of ibrutinib (from 0 to 20 μM) and of ibrutinib (from 0 to 20 μM)+5FU (200 μM) and their viability was evaluated at the end of the incubation using the calcein assay.
As can be seen from the graphs reported in FIG. 3, concomitant administration of 200 μM 5FU and ibrutinib re-sensitize resistant HCT116p53KO cells to the cytotoxic effect of 5FU, starting from the concentration of 100 nM, to achieve a maximal effect at 20 μM. In contrast, the addition of ibrutinib to 5FU in cell cultures of HCT116 (sensitive) has no additive effect on the response to chemotherapy.
In FIG. 4 HCT116 drug-sensitive cells, HCT116 overexpressing p65BTK (after transfection with a plasmid encoding p65BTK, p65 BTK is in the same amount as in HCT116p53KO drug-resistant cells) and HCT116p53KO drug-resistant cells were left untreated or treated with 200 μM 5FU, 20 μM ibrutinib or the combination of the two. Viability was evaluated after 72 hours of incubation by calcein assay. 100% represents the percentage of living cells at day 0, before starting the treatment with the drugs. It is clearly shown that HCT116 drug-sensitive cells overexpressing p65BTK, similarly to HCT116p53KO, become resistant to the cytotoxic effect of 5FU and that the resistance in both cases is reversed by concomitant addition of 20 μM ibrutinib, further validating the concept that to abolish 5FU-resistance is necessary to block p65BTK by ibrutinib.

### Example 4

### Treatment with Ibrutinib and Fluorouracil: Lower Amounts of Chemotherapeutic Drug are Sufficient

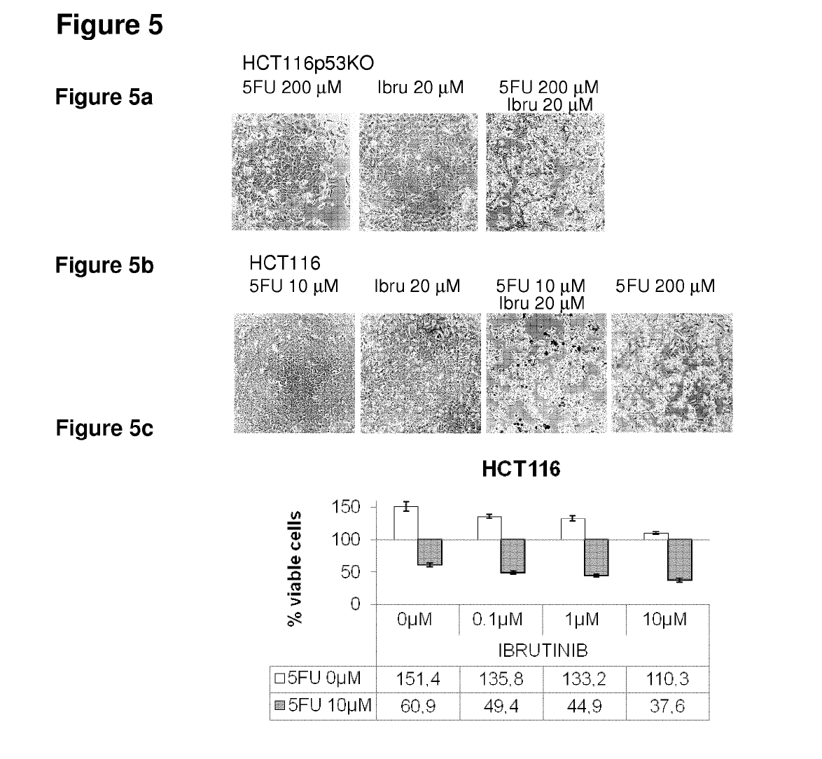

In FIG. 5 is depicted the synergistic effect of singularly ineffective concentrations of 5FU and ibrutinib in drug-resistant and -sensitive colon carcinoma cells. FIG. 5a shows representative photographs of drug-resistant HCT116p53KO cells taken at 72 hours of incubation in presence of 200 μM 5FU, 20 μM ibrutinib, 200 μM 5FU+20 μM ibrutinib: it is evident that only in the latter experimental condition few cells are still alive and attached to the plate and that the vast majority of the cells are dead. FIG. 5b shows representative photographs of sensitive HCT116 cells taken at 72 hours of incubation in presence of non-effective concentrations of 5FU and ibrutinib. When the two drugs are used singularly (10 μM 5FU and 20 μM ibrutinib) no toxicity is evident, whereas when the two drugs are used in combination (10 μM 5FU+20 μM ibrutinib) all the cells are detached from the culture dish because are dead. The fourth picture, shown for comparison, is of a culture dish where HCT116 have been treated with the maximally effective concentration of 200 μM. FIG. 5c show a dose-response curve, where HCT116 cell lines were grown in vitro for 72 hs in the presence of different low concentrations of ibrutinib (from 0 to 10 μM) and of ibrutinib (from 0 to 10 μM)+a low dose of 5FU (10 μM); their viability was evaluated at the end of the incubation using the crystal violet staining method. 100% represents the percentage of living cells at day 0, before starting the treatment with the drugs. 100% represent the percentage of living cells at day 0, before starting the treatment with the drugs. As can be seen, an ibrutinib concentration as low as 0.1 μM synergize with a low dose of 5FU and increases the percentage of cell responding to the treatment.
These data clearly show that combining singularly ineffective concentrations of 5FU and ibrutinib not only sensitize drug-resistant cells to chemotherapy but also allows to reduce the concentration of 5FU necessary to kill sensitive cells.

### Example 5

### Effect of Ibrutinib on the Response to Other Anticancer Drugs Commonly Used for CRC Therapy (Oxaliplatin, Targeted Therapy)

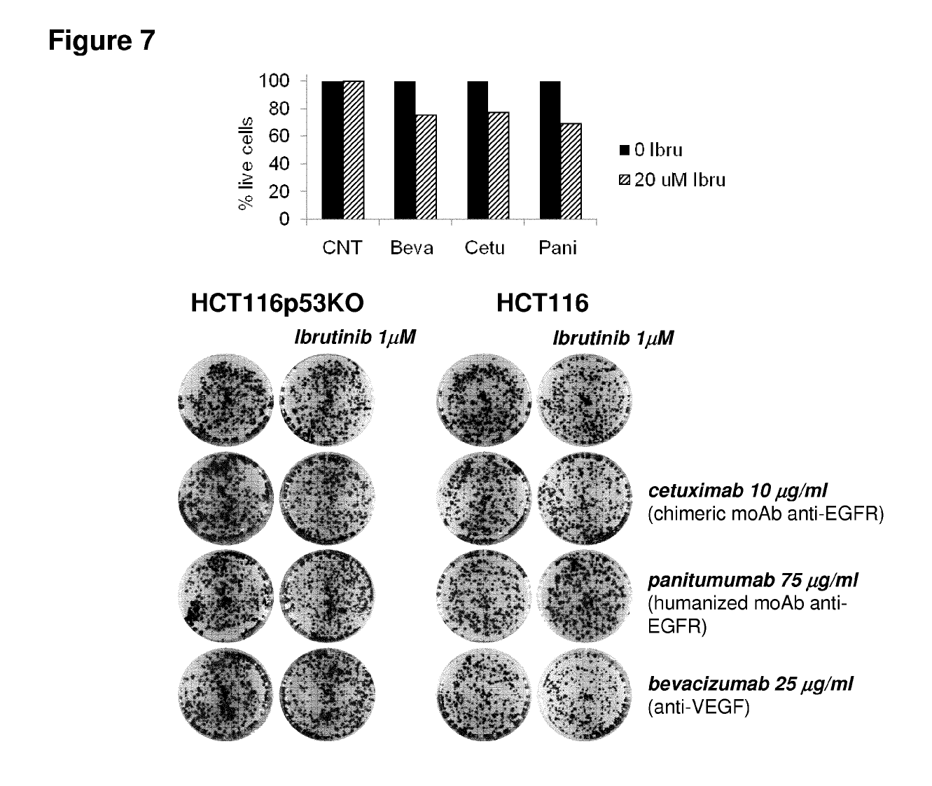

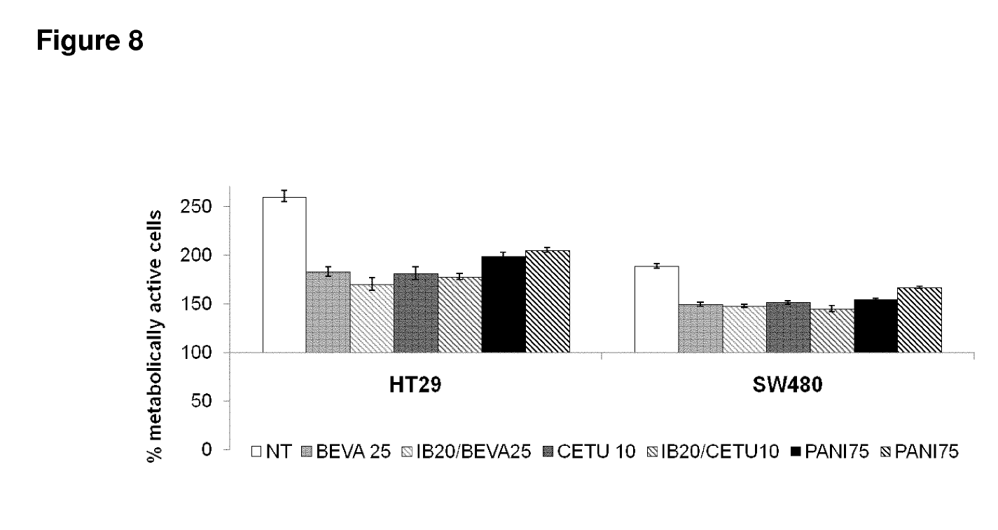

In FIG. 6 drug-resistant SW480 colon cancer cells were treated for 72 hs in absence of any drug or in presence of ibrutinib 20 μM; 5FU 200 μM; the combination of ibrutinib 20 μM+5FU 200 μM; oxaliplatin 20 μM; the combination of ibrutinib 20 μM+oxaliplatin 20 μM. At the end of the treatment cell viability was evaluated by calcein assay. 100% represents the percentage of living cells at day 0, before starting the treatment with the drugs. As evident, Ibrutinib addition was able to revert the resistance only to 5FU but not to oxaliplatin.
In FIG. 7 HCT116p53KO and HCT116 cell lines were grown in vitro and subjected to the combined treatment of ibrutinib and three different monoclonal antibodies currently used in anti-cancer therapy for targeting EGFR (cetuximab, panitumumab) and VEGF (bevacizumab), both in short term (upper panel) and in long term (lower panel) experiments.
In the upper part of FIG. 7 the graph shows that the combined treatment with 20 μM ibrutinib and these “targeted” drugs does not revert the resistance of HCT116p53KO cells, as assessed by Trypan blue staining 72 hs after the combined treatment. As shown in the lower panel, also the continuous exposure for 10-12 days to the combined treatment does not re-sensitize resistant cells to targeted therapy.
In FIG. 8 drug-resistant HT29 and SW480 colon cancer cell lines were left untreated or treated with 25 μg/ml bevacizumab or 10 μg/ml cetuximab or 75 μg/ml panitumumab alone or in combination with 20 μM ibrutinib. Percentage of viable cells was evaluated after 72 hs of treatment by MTT assay (a colorimetric assay based on the reduction of tetrazolium salts by enzymes, functioning only in metabolically active, viable cells). 100% represent the percentage of living cells at day 0, before starting the treatment with the drugs. As evident from the graph the combination of ibrutinib with targeted drugs does not decrease the number of live cells as compared to the single treatment, thus indicating that ibrutinib do not abolish the resistance of colon cancer cells to targeted therapy.

### Example 6

### In Vivo Treatment with Ibrutinib and Fluorouracil

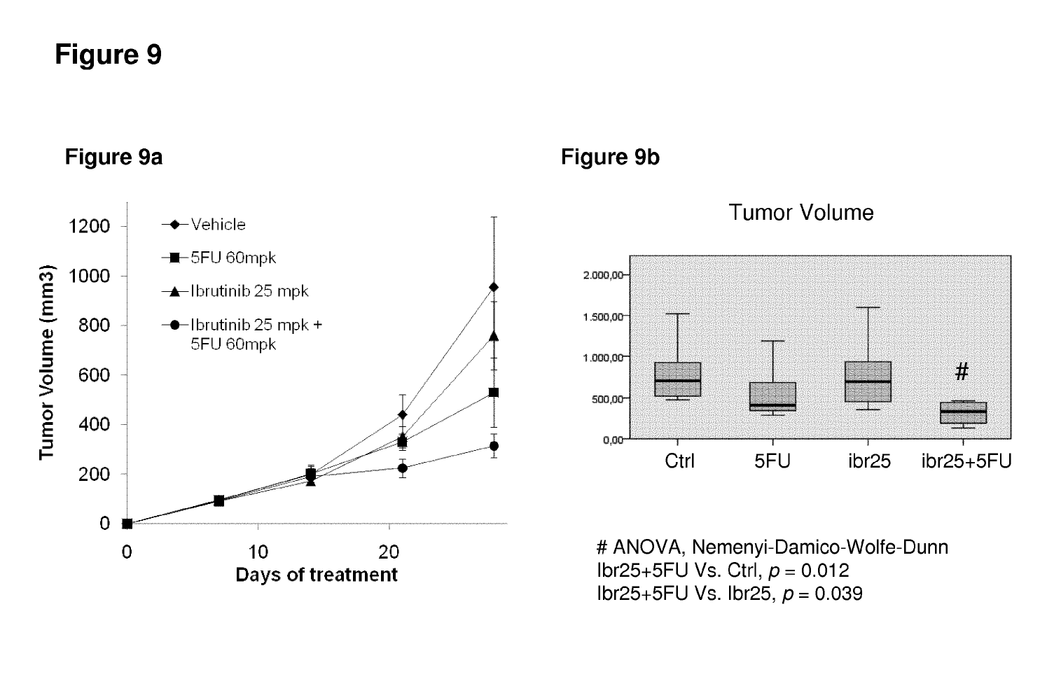

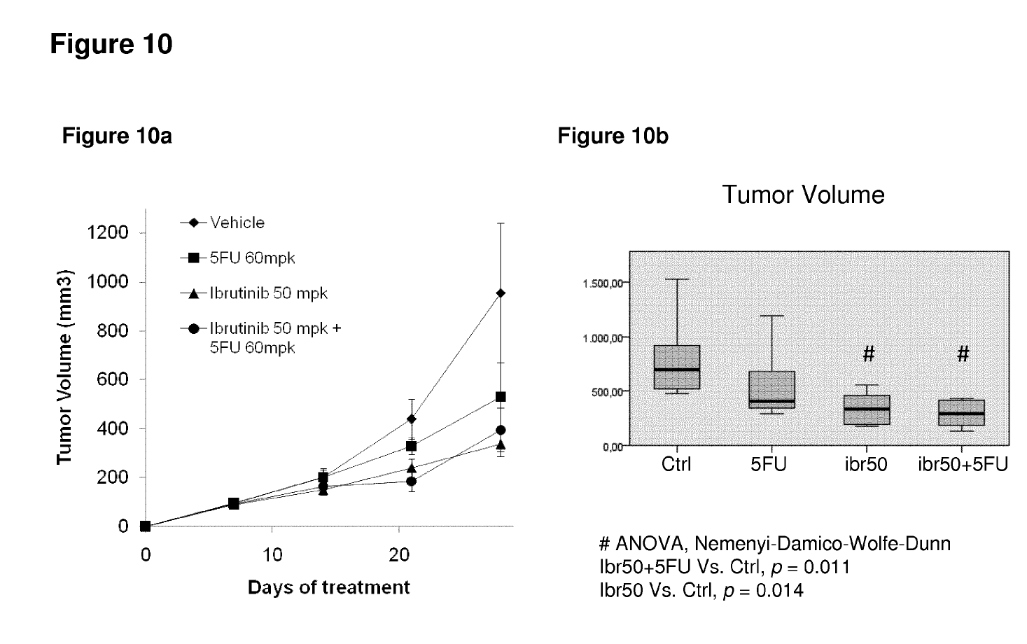

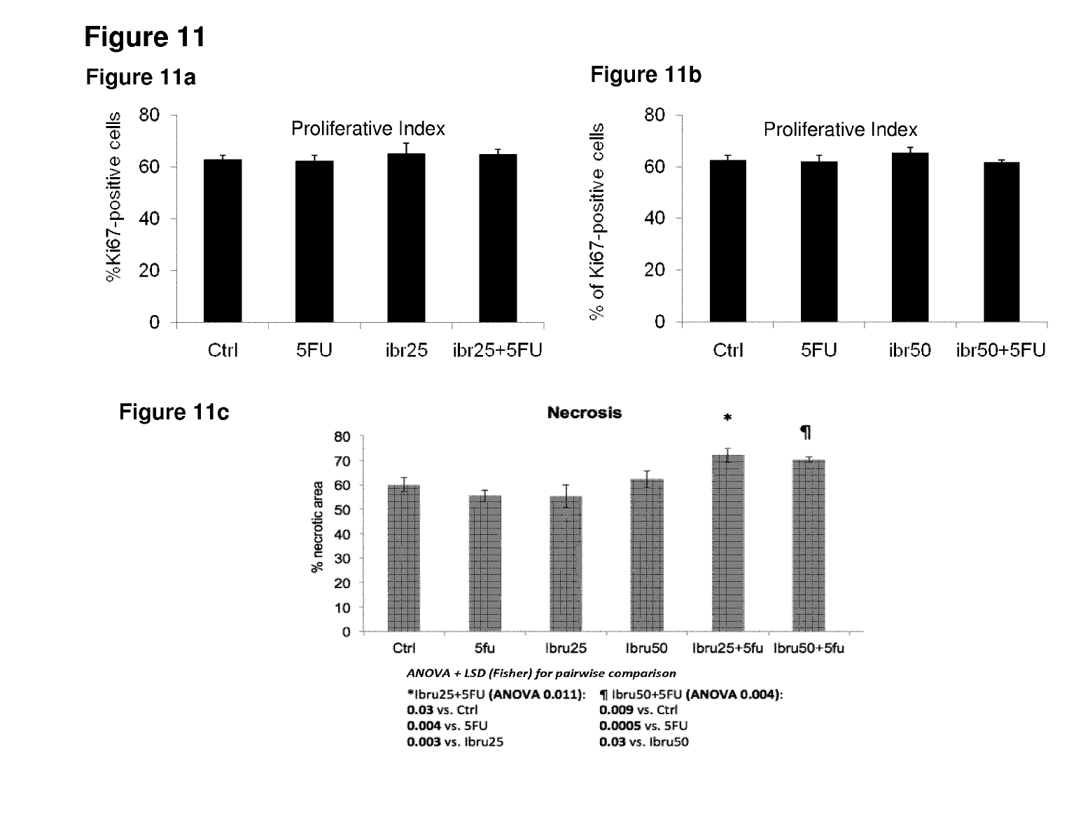

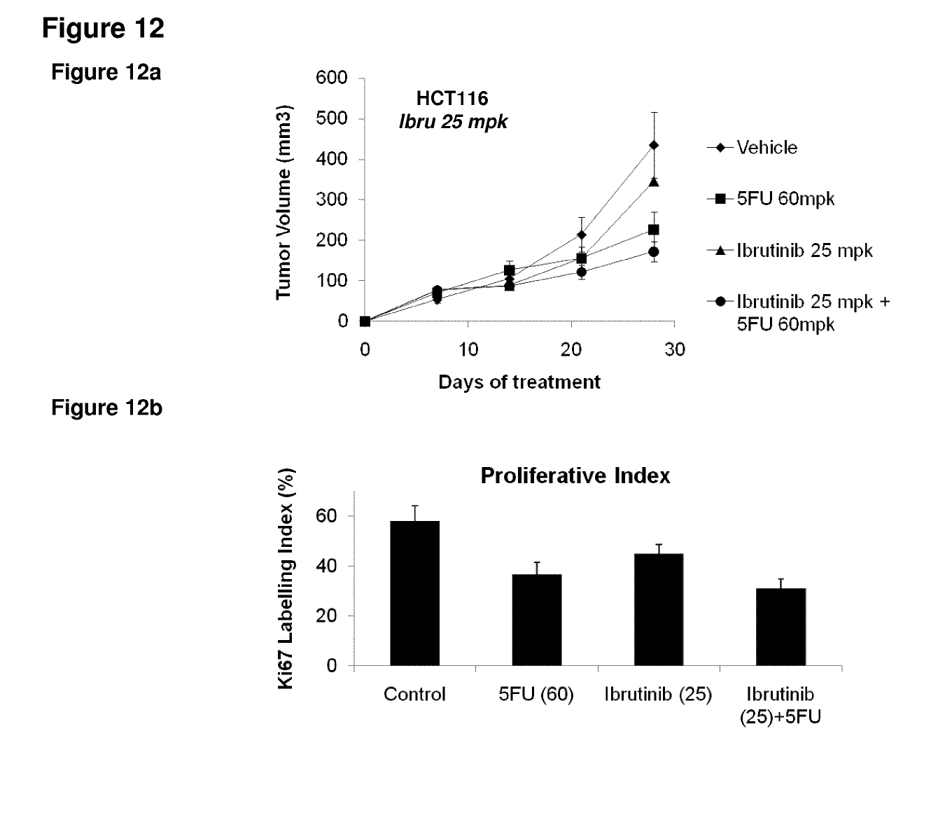

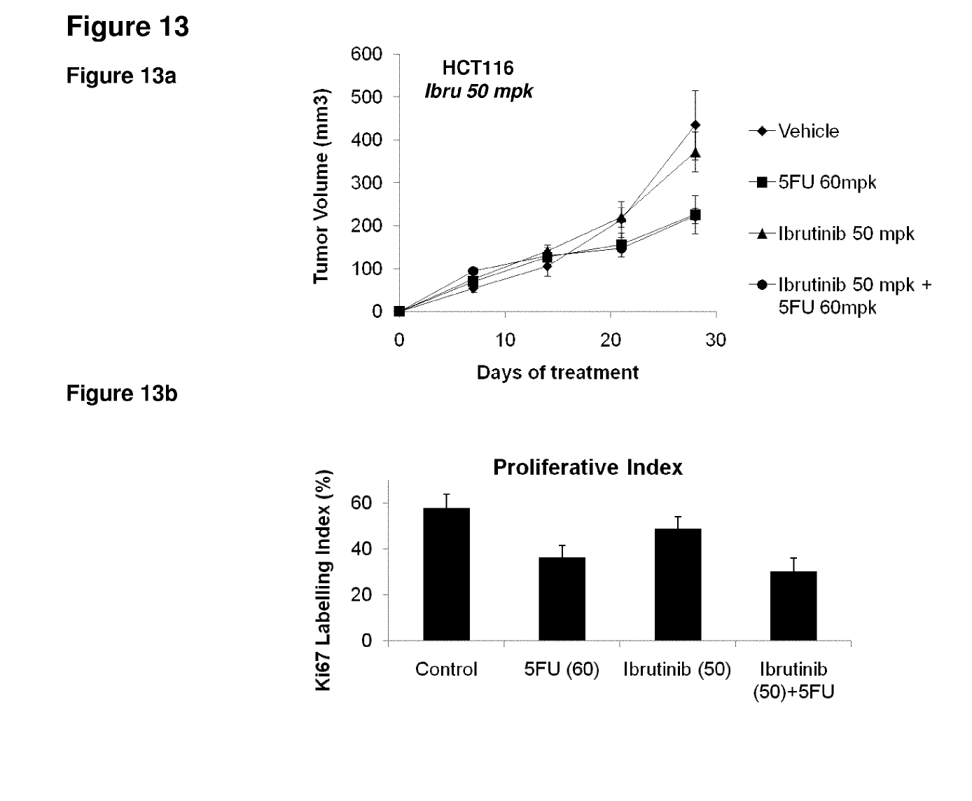

Tumors were established by injecting s.c. 1×10⁶ cells (in 100 μL of a 50% PBS and 50% Matrigel solution), HCT116p53KO cells into the left flanks and HCT116 into the right flanks, of 5 to 7 weeks old female CD-1 nude mice (n=8 mice/group). When HCT116p53KO tumors reached the average volume of 100 mm³ (day 8 postengraftment), animals were randomized and given vehicle, 5FU [via intraperitoneal (i.p.) injection, 60 mg/kg, twice a week], ibrutinib [via oral gavage, 25 mg/kg (mpk) or 50 mpk once day for 5 days a week], or a combination thereof. 5FU treatment started at day 9 post-engraftment, whereas ibrutinib treatment started at day 8 post-engraftment. Control mice received i.p. injections of vehicle (0.9% NaCl solution) with the same schedule of the other groups. Tumors were measured with caliper once a week. Statistical significance was determined with a Kruskal-Wallis non parametric test (normal distribution not assumable), followed by Nemenyi-Damico-Wolfe-Dunn test for multiple pairwise comparisons between groups. In all cases, a P value <0.05 was considered as significant
As can be seen from the graph reported in FIG. 9a, after 28 days of treatment the average volume of the tumor masses formed by xenografted drug-resistant HCT116p53KO amounts to ≈955 mm³ for the group of untreated mice (vehicle), to ≈760 mm³ for the group treated with ibrutinib (25 mpk) alone, to ≈530 mm³ for the group treated with 5FU alone and to ≈315 mm³ for the group treated with the combination of ibrutinib and 5FU. As illustrated in FIG. 9b statistical analysis shows that the reduction in the tumor volume is not significant when comparing the group of mice treated with 5FU alone vs the untreated group but it become significant when comparing the group of mice treated with 5FU+ibrutinib vs. the untreated group and vs. the group treated with ibrutinib alone.
Data reported in FIG. 10a show that doubling the dose of ibrutinib (50 mpk) administered to mice do not further decrease the volume of the tumors of the group of mice treated with the combination 5FU+ibrutinib (compared to the group treated with 5FU+Ibru 25 mpk). However, the treatment with the high dose of ibrutinib has an anti-tumoral effect on its own: the statistical analysis reported in FIG. 10b shows that, when compared to the untreated group, the reduction in the tumor volume obtained administering to the mice ibrutinib 50 mpk is as significant as that obtained administering the combination 5FU+ibrutinib 50 mpk or combination 5FU+ibrutinib 25 mpk.
These data show that the combined treatment with ibrutinib and fluorouracil has a synergistic effect on the tumor volume reduction when low doses of ibrutinib are administered together with chemotherapy to mice bearing drug-resistant xenografts and that an higher dosage of ibrutinib has an anti-tumoral effect on its own. The graphs reported in FIG. 11 show the results of the analysis performed on the tumoral tissues excised from the mice at the end of the different treatments. To evaluate the proliferative index tissue sections were stained with antibodies against Ki67, a marker of proliferating cells. Percentage of Ki67-positive cells was quantified by Scanscope dedicated software upon digital acquisition of Ki67-stained, hematoxylin-eosin counter-stained slides (FIGS. 11a and b). Using another feature of the dedicated software also the percentage of necrotic area was calculated on the same slides (FIG. 11c).
These data show that both, at low and high doses, neither ibrutinib alone nor the combination ibrutinib+5FU affect the proliferation of tumor cells whereas the combination increases significantly the extension of the necrotic area.
The graph in FIG. 12a, represents the growth curve of tumors derived from HCT116 xenografted in CD1 mice after 28 days of treatment with vehicle alone, ibrutinib (25 mpk), 5FU (60 mpk) and ibrutinib (25 mpk)+5FU (60 mpk). By the end of the treatment the average volume of the tumor masses formed by xenografted drug-sensitive HCT116 cells amounts to ≈435 mm³ for the group of untreated mice (vehicle), to ≈347 mm³ for the group treated with ibrutinib (25 mpk) alone, to ≈226 mm³ for the group treated with 5FU alone and to ≈172 mm³ for the group treated with the combination of ibrutinib and 5FU. The difference in tumor size is not statistically significant between vehicle-treated and ibrutinib-treated groups nor between 5FU-treated and 5FU+ibrutinib-treated groups. Reduction in the tumor volume is in fact significant only when comparing the group of mice treated with 5FU±ibrutinib vs the untreated (or ibrutinib-treated) group.
Data plotted in the graph of FIG. 12b represent the percentage of proliferating cells in the tumoral tissues excised from the mice at the end of the treatments shown above: 5FU treatment±ibrutinib significantly reduces the percentage of proliferating cells compared to either vehicle or ibrutinib alone.
The graph in FIG. 13a, represents the growth curve of tumors derived from HCT116 xenografted in CD1 mice after 28 days of treatment with vehicle alone, ibrutinib (50 mpk), 5FU (60 mpk) and ibrutinib (50 mpk)+5FU (60 mpk). By the end of the treatment the average volume of the tumor masses formed by xenografted drug-sensitive HCT116 cells amounts to ≈435 mm³ for the group of untreated mice (vehicle), to ≈372 mm³ for the group treated with ibrutinib (50 mpk) alone, to ≈226 mm³ for the group treated with 5FU alone and to ≈223 mm³ for the group treated with the combination of ibrutinib and 5FU. The difference in tumor size is not statistically significant between vehicle-treated and ibrutinib-treated groups nor between 5FU-treated and 5FU+ibrutinib-treated groups. Reduction in the tumor volume is in fact significant only when comparing the group of mice treated with 5FU±ibrutinib vs the untreated (or ibrutinib-treated) group.
Data plotted in the graph of FIG. 13b represent the percentage of proliferating cells in the tumoral tissues excised from the mice at the end of the treatments shown above: 5FU treatment±ibrutinib significantly reduces the percentage of proliferating cells compared to either vehicle or ibrutinib alone.
All together these data show that the growth-reducing and anti-proliferative effects of 5FU on sensitive xenografts are achieved independently from the addition of ibrutinib, which by itself is ineffective in these kind of tumors.

### Example 7

### Effects of the Combined Treatment of Ibrutinib and 5FU on Drug-Resistant Epithelial Cancer Cells Derived from Tumours Other than Colon

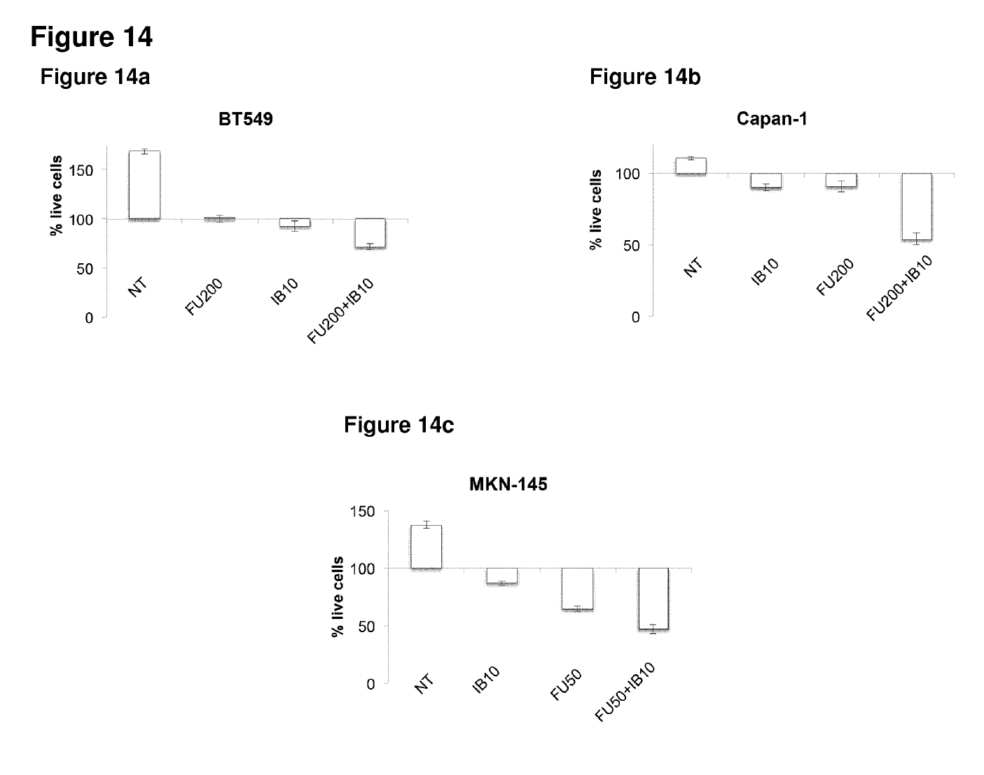

In FIG. 14a drug-resistant BT549 breast cancer cells were treated for 72 hs in absence of any drug or in presence of ibrutinib 10 μM; 5FU 200 M; the combination of ibrutinib 10 μM+5FU 200 μM. At the end of the treatment cell viability was evaluated by MTT assay. As evident, Ibrutinib addition was able to sensitize the cells to 5FU. In FIG. 14b drug-resistant Capan-1 pancreatic cancer cells were treated for 72 hs in absence of any drug or in presence of ibrutinib 10 μM; 5FU 200 μM; the combination of ibrutinib 10 μM+5FU 200 μM. At the end of the treatment cell viability was evaluated by MTT assay. As evident, Ibrutinib addition was able to revert the resistance to 5FU. In FIG. 14c MKN-145 stomach cancer cells were treated for 72 hs in absence of any drug or in presence of ibrutinib 10 μM; 5FU 50 μM; the combination of ibrutinib 10 μM+5FU 50 μM. At the end of the treatment cell viability was evaluated by MTT assay. As evident, Ibrutinib addition was able to further decrease cell viability compared to 5FU alone.
From the above description and the above-noted examples, the advantage attained by the product described and obtained according to the present invention are apparent.

1.-22. (canceled)

23. A method of treating a cancer patient by administration of a therapeutically effective amount of combination therapy comprising a small molecule BTK inhibitor selected form the group consisting of:
ibrutinib in a range from 1 to 60 mg/kg, HM-71224, BGB-3111, CG-036806, CC-292, ACP-196, GDC-0834, ONO-4049, RN-486, SNS-062, TAS-5567, AVL-101, AVL-291, PCI-45261, HCI-1684 and PLS-123 and
fluorouracil concomitantly,
wherein the BTK inhibitor is not adjuvant therapy, wherein the cancer is a solid tumor, wherein the dose of fluorouracil is in a range from 10 to 60 mg/Kg and wherein the fluorouracil dose, the BTK inhibitor dose or both are less than the dose employed for the corresponding monotherapy.

24. The method according to claim 23, wherein the BTK inhibitor is ibrutinib or CC-292.

25. The method according to claim 24, wherein the dose of ibrutinib is in the range from 5 to 50 mg/kg, such as 10 to 50 mg/Kg, in particular 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 45, 46, 47, 48 and 49 mg/Kg per administration.

26. The method according to claim 24, wherein the effective dose of ibrutinib is in the range of from 25 mg/day to 840 mg/day, for example 50, 75, 100, 125, 150, 175, 200, 225, 250, 270, 275, 300, 325, 350, 375, 400, 425, 450, 500, 525, 540, 550, 575 and 600 mg/day.

27. The method according to claim 23, wherein the amount of fluorouracil is in the range from 15 to 50 mg/Kg, in particular 16, 17, 18, 19, 20, 21, 22, 23, 24, 25, 26, 27, 28, 29, 30, 31, 32, 33, 34, 35, 36, 37, 38, 39, 40, 41, 42, 43, 45, 46, 47, 48 and 49 mg/Kg per administration.

28. The method according to claim 23, wherein the effective dose of fluorouracil is in the range of from 400 mg/m² to 600 mg/m².

29. The method according to claim 23, wherein the cancer is p53 defective or deficient.

30. The method according to claim 23, wherein the cancer is drug resistant.

31. The method according to claim 30, wherein the drug resistance is to fluorouracil.

32. The method according to claim 30, wherein the drug resistance is to a BTK inhibitor.

33. The method according to claim 23, wherein the cancer is an epithelial cancer, for example selected from the group consisting of such as colorectal cancer, hepatoma (liver cancer), prostate cancer, stomach cancer, pancreatic cancer, breast cancer, ovarian cancer, thyroid cancer, renal cancer, bladder cancer, head and neck cancer or lung cancer.

34. The method according to claim 23, wherein the cancer is metastatic cancer.

35. A method of treating a fluorouracil resistant cancer patient comprising administering a therapeutically effective amount of a BTK inhibitor selected from the group consisting of ibrutinib in a range from 1 to 60 mg/kg, HM-71224, BGB-3111, CG-036806, CC-292, ACP-196, GDC-0834, ONO-4049, RN-486, SNS-062, TAS-5567, AVL-101, AVL-291, PCI-45261, HCI-1684 and PLS-123, for sensitizing the patient to treatment with fluorouracil in a range from 10 to 60 mg/Kg, wherein the cancer is a solid tumor.

36. A combination therapy comprising a BTK inhibitor selected from the group consisting of ibrutinib in a range from 1 to 60 mg/kg, HM-71224, BGB-3111, CG-036806, CC-292, ACP-196, GDC-0834, ONO-4049, RN-486, SNS-062, TAS-5567, AVL-101, AVL-291, PCI-45261, HCI-1684 and PLS-123 and fluorouracil in a range from 10 to 60 mg/Kg for concomitant administration, wherein the BTK inhibitor is not adjuvant therapy, for use in the treatment of a solid tumor, such as cancer of epithelial origin wherein the cancer is drug resistant.

37. Use of a BTK inhibitor selected from the group consisting of ibrutinib in a range from 1 to 60 mg/kg, HM-71224, BGB-3111, CG-036806, CC-292, ACP-196, GDC-0834, ONO-4049, RN-486, SNS-062, TAS-5567, AVL-101, AVL-291, PCI-45261, HCI-1684 and PLS-123 and fluorouracil in a range from 10 to 60 mg/Kg in the manufacture of a combination therapy for concomitant administration, for the treatment of a solid tumor, such as cancer of epithelial origin wherein the cancer is drug resistant.
